(19) United States
(12) Patent Application Publication
(10) Pub. No.: US 2021/0106791 A1
(43) Pub. Date: Apr. 15, 2021

(54) Guide Wire for Medical Magnetic Resonance Applications

(71) Applicant: EPflex Feinwerktechnik GmbH, Dettingen/Erms (DE)

(72) Inventor: Bernhard UIHLEIN, Dettingen (DE)

(21) Appl. No.: 16/977,348

(22) PCT Filed: Feb. 26, 2019
(86) PCT No.: PCT/EP2019/054742

§ 371 Date: Sep. 1, 2020

(30) Foreign Application Priority Data
Mar. 1, 2018 (DE) 10 2018 203 102.9

(51) Int. Cl. A61M 25/09; A61M 25/01; A61M 25/00

(52) U.S. Cl. CPC A61M 25/09; A61M 25/0127; A61M 25/0026; A61M 2025/0915; A61M 2025/0039; A61M 2025/09141; A61M 25/0158

(57) ABSTRACT

A guide wire configured for use in medical magnetic resonance applications includes a multi-lumen wire composed of an electrically nonconductive plastic material. The wire extends continuously from a proximal end region to a distal end region of the guide wire and has at least two separate, axially extending hollow channels, and/or at least two coaxial wires arranged coaxially one inside another and extending continuously from a proximal end region to a distal end region of the guide wire. An axially alternating sequence of rod-shaped, elastic stiffening pieces composed of an electrically conductive, nonmagnetic material having higher bending stiffness than the plastic material of the multi-lumen wire, and electrically nonconductive spacer pieces, is arranged in at least one of the hollow channels. At least one of the coaxial wires is constructed from an axially alternating sequence of rod-shaped, elastic stiffening pieces composed of an electrically conductive, nonmagnetic material and electrically nonconductive spacer pieces.

### BACKGROUND AND SUMMARY OF THE INVENTION

The invention relates to a guide wire configured for use in medical magnetic resonance (MR) applications.

Guide wires are used in various embodiments in invasive medical technology, in particular for insertion into human or animal body passageways, in order to guide subsequent introduction of a catheter tube or the like by means of the inserted guide wire. For applications in magnetic resonance tomography (MRT) or nuclear magnetic resonance (NMR) applications, or MR applications for short, particular requirements are made of the guide wire. The traditional guide wires are generally not suitable for this, said guide wires including a wire that is composed of an electrically conductive metal material, such as a high-grade steel material or a nickel-titanium (NiTi) alloy, and extends continuously from a proximal end region to a distal end region of the guide wire. Such a continuous metal wire can result, inter alia, in disturbing artefacts in MR imaging and in undesired heating effects as a result of inductive heating in the magnetic field. Guide wires configured for use in medical MR applications have already been proposed on various occasions as a remedy; see, for example, the laid-open publications DE 100 29 738 A1, US 2005/0064223 A1 and DE 10 2011 081 445 A1 and also the patent publications EP 0 864 102 B1 and DE 10 2005 022 688 B4.

The laid-open publication DE 10 2007 016 674 A1 discloses a guide wire that is formed axially from successive sections, the material of which consists alternately of electrical conductors, such as noble metals and NiTi alloys, and insulators, such as polyurethane, polyethylene, polymers and glass fibers, wherein the successive sections are adhesively bonded, welded or screwed to one another. In the interior of the guide wire, a hollow channel filled with a gel or a contrast liquid can be provided; in addition, one or more hollow channels functioning as working channels can be formed.

The laid-open publication WO 2007/000148 A2 discloses a guide wire suitable for MR that consists of one or more rod-shaped bodies and a non-ferromagnetic matrix material, which encloses and/or adhesively bonds together the rod-shaped body (bodies). The rod-shaped bodies consist of one or more nonmetallic filaments and a non-ferromagnetic material, which encloses and/or adhesively bonds together the filament(s) and is doped with MR marker particles. The filaments consist of plastic and/or glass fiber, and the matrix material consists of epoxy resin.

The laid-open publication WO 2009/141165 A2 discloses a similar guide wire constructed from a central rod-shaped body and six rod-shaped bodies arranged at a distance around the latter and having a smaller diameter than the central rod-shaped body, wherein the rod-shaped bodies are embedded into an enveloping matrix. The enveloping matrix consists of a thermoplastic elastomer. The rod-shaped bodies are formed from a matrix material containing nonmetallic filaments, wherein a ceramic or a plastic is used as matrix material. In addition, the rod-shaped bodies are doped with MR marker particles at their surface.

The technical problem addressed by the invention is that of providing a guide wire of the type mentioned in the introduction which, by comparison with the prior art mentioned above, affords advantages with regard to its behavior under MR conditions, its bending behavior or its flexibility and/or with regard to a comparatively low production outlay.

The invention solves this problem by providing an inventive guide wire having the specific features as detailed below.

In accordance with a first aspect of the invention, the inventive guide wire comprises a multi-lumen wire composed of an electrically nonconductive plastic material, said wire extending continuously from a proximal end region to a distal end region of the guide wire and having at least two separate, axially extending hollow channels. An axially alternating sequence of rod-shaped, elastic stiffening pieces composed of an electrically conductive, nonmagnetic material having higher bending stiffness than the plastic material of the multi-lumen wire and electrically nonconductive spacer pieces is arranged in at least one of the hollow channels.

In accordance with a further aspect of the invention, which can be realized in addition or as an alternative to the above, first-mentioned aspect the inventive guide wire has at least two wires or individual wires, referred to as coaxial wires in the present case, arranged coaxially one inside another and extending continuously from a proximal end region to a distal end region of the guide wire. At least one of the coaxial wires is constructed from an axially alternating sequence of rod-shaped, elastic stiffening pieces composed of an electrically conductive, nonmagnetic material and electrically nonconductive spacer pieces. In corresponding embodiments, all the coaxial wires are constructed in this way; in alternative embodiments, one or more remaining coaxial wires are formed by said multi-lumen wire or by some other continuous wire that is not electrically conductive throughout and can consist e.g. of an electrically nonconductive solid material composed of plastic.

In this regard, e.g. an innermost, central coaxial wire can be formed as such a multi-lumen wire or as a core wire composed of an electrically insulating solid material or composed of alternately strung together stiffening and spacer pieces composed of respective solid material, and/or at least one surrounding coaxial wire can in turn be formed as such a multi-lumen wire in a hollow tube embodiment, in which the hollow channel(s) is/are situated in the interior of the hollow tube material, which is ring-shaped in cross section, or as a hollow tube wire composed of an electrically insulating hollow tube material or composed of alternately strung together stiffening and spacer pieces composed of respective hollow tube parts or ring parts. Consequently, depending on the embodiment, the coaxial wire construction includes no, one or a plurality of multi-lumen wires of the type considered in the present case.

It goes without saying that the extent of the multi-lumen wire and of its hollow channels and/or of the coaxial wires from the proximal end region to the distal end region of the guide wire constitutes the very largest portion of the total extent of the guide wire, without excluding embodiments in which a short, different distal terminating section of the guide wire is adjacent to the distal end of the multi-lumen wire and/or of the coaxial wires and/or a short, proximal terminating region of the guide wire is adjacent to the proximal end, as known per se for guide wire designs. The distal terminating region can include e.g. a flexurally more pliant, distal end tip and/or a so-called J-tip of the guide wire. The proximal terminating region can include e.g. coupling means for coupling the guide wire to an operating handle or the like. Preferably, the extent of the multi-lumen wire and of its hollow channels and/or of the coaxial wires from the proximal to the distal end region of the guide wire comprises that principal length portion of the guide wire which is located within a body tissue passageway during use and is exposed to the MR conditions there during MR applications, excluding said distal terminating region.

The guide wire according to the invention affords advantages with regard to its behavior under MR conditions, its bending behavior or its flexibility and/or a low production outlay. Since the multi-lumen wire and the spacer pieces in the relevant hollow channel(s) and/or as parts of the coaxial wire(s) are electrically nonconductive, long electrically conductive sections that could result in undesired heating effects can be avoided in the case of the guide wire according to the invention. The use of the rod-shaped, elastic stiffening pieces composed of electrically conductive, nonmagnetic material makes it possible to optimize the behavior under MR conditions and the bending behavior or the flexibility of the guide wire. The production of the guide wire comprising the multi-lumen wire and the stiffening pieces and the spacer pieces in the hollow channel of said wire and/or with the coaxial wire construction comprising a plurality of coaxial wires lying coaxially one inside another, at least one of which is constructed from the stiffening pieces and the spacer pieces, generally requires only a comparatively low outlay.

In one development of the invention, each stiffening piece has a greater length than each spacer piece. This is preferred for many applications. In this case, the bending behavior, i.e. the elastic bending capability or the elastic bending stiffness, of the guide wire can substantially be determined by the stiffening pieces, and the shorter spacer pieces by comparison therewith preferably serve primarily for electrically interrupting or insulating successive stiffening pieces.

In one development of the invention, each stiffening piece has a length of between 1 cm and 15 cm. This relatively short length in comparison with typical total lengths of guide wires makes it possible to reliably avoid any risk of overheating effects as a result of inductive heating of the stiffening pieces under MR conditions. On the other hand, a length of the stiffening pieces that is not excessively short is generally advantageous with regard to the production outlay.

In one development of the invention, the length of each spacer piece is in the range of between 0.1 mm and 5 cm. This dimensioning of the spacer pieces is advantageous for many applications. A short length of the spacer pieces of e.g. at most approximately 1 mm or at most approximately 0.5 cm or at most approximately 1 cm is often striven for.

In advantageous embodiments, the length of the respective stiffening piece is greater than the length of the respective spacer piece by at least a factor of five, in appropriate realizations by at least a factor of ten.

In one development of the invention, the stiffening pieces and the spacer pieces are arranged loosely successively, i.e. are inserted loosely into the respective hollow channel of the multi-lumen wire or chained together loosely to form the relevant coaxial wire. This realization is very advantageous both with regard to production outlay and with regard to functional reliability. The loose arrangement of the stiffening pieces and of the spacer pieces obviates any outlay for producing corresponding connections between the stiffening pieces, on the one hand, and the spacer pieces, on the other hand, and/or between the multi-lumen wire material, on the one hand, and the stiffening pieces and/or the spacer pieces, on the other hand. Accordingly, during use there is also no risk at all of corresponding connections breaking or detaching. Moreover, the multi-lumen wire, the stiffening pieces and the spacer pieces can each be prefabricated separately, and the stiffening pieces and the spacer pieces can then be inserted alternately into the respective hollow channel or be strung together to form the relevant coaxial wire, or be threaded onto an existing, radially inner guide wire part.

In one development of the invention, a continuous tension rod composed of an electrically nonconductive plastic material is arranged at least in one of the hollow channels of the multi-lumen wire. In one development of the invention, at least one of the coaxial wires is formed by a continuous tension rod composed of an electrically nonconductive plastic material. In these embodiments of the guide wire, the tension rod, which is designated thus in the present case for this reason, can provide the required tensile strength of the guide wire entirely or in any case predominantly. For this purpose, it consists of a material having a suitable tensile strength or high strength, as known per se for such applications in guide wires. In corresponding embodiments, the tension rod has a low bending stiffness in comparison with the stiffening pieces, such that the bending stiffness or the bending capability of the guide wire is primarily determined by the stiffening pieces. Alternatively, the tension rod can be embodied such that it makes a not inconsiderable contribution, together with the stiffening pieces, to the desired bending stiffness, i.e. bending capability or flexibility, of the guide wire. Since the tension rod is electrically nonconductive, it does not cause any disturbing or undesired effects under MR conditions despite its continuous extent from the proximal to the distal end region of the guide wire. The tension rod can be produced simultaneously with the multi-lumen wire or can be produced separately therefrom and subsequently be inserted into the relevant hollow channel. It can relieve the multi-lumen wire of tensile force loads; i.e. the multi-lumen wire in this case need not be designed in regard to the tensile strength required for the guide wire.

In one configuration of the invention, the hollow channels in the multi-lumen wire include a central hollow channel, in which the continuous tension rod composed of electrically nonconductive plastic material is arranged. In an alternative configuration of the invention, the coaxial wires include a central coaxial wire, which is formed by the continuous tension rod composed of an electrically nonconductive plastic material. The central arrangement of the tension rod determining the tensile strength of the guide wire is advantageous for many applications.

In one development of the invention, the hollow channels in the multi-lumen wire include a central hollow channel, in which the alternating sequence of the stiffening pieces and spacer pieces is arranged. In an alternative development of the invention, the coaxial wires include a central coaxial wire, which is formed by the alternating sequence of the stiffening pieces and spacer pieces. These embodiments can afford advantages e.g. with regard to largely direction-independent flexural strength for many applications, including the cases in which no central tension rod is provided.

In one development of the invention, for a plurality of the hollow channels and/or coaxial wires the alternating sequence of the stiffening pieces and spacer pieces is arranged and the stiffening pieces and spacer pieces in a first of said hollow channels and/or coaxial wires are arranged axially offset relative to the stiffening pieces and spacer pieces in a second of said hollow channels and/or coaxial wires. This can be advantageous in order to achieve a bending stiffness that is as uniform as possible, i.e. a bending behavior that is as uniform as possible, of the guide wire along the axial extent thereof. Moreover, the behavior of the guide wire under MR conditions can thereby be optimized as necessary.

In one configuration of the invention, the spacer pieces are arranged in a manner axially free of overlap in the guide wire, i.e. in the hollow channels and/or for the coaxial wires. This means that at any arbitrary point along the axial extent of the guide wire in the cross section of the guide wire there is at most one spacer piece present, even if a plurality of hollow channels and/or coaxial wires are filled or constructed by the alternating sequence of stiffening pieces and spacer pieces. Conversely, this means that at any point along the axial extent of the guide wire in cross section at least one of the stiffening pieces is present. These properties are advantageous with regard to behavior under MR conditions and bending behavior for many applications.

In one development of the invention, the hollow channels in the multi-lumen wire include a plurality of eccentric hollow channels, in which the alternating sequence of the stiffening pieces and spacer pieces is arranged. This can be advantageous in particular for achieving a bending behavior of the guide wire that is as uniform as possible on all sides, particularly if, in corresponding embodiments, the eccentric hollow channels are arranged around a longitudinal central axis of the guide wire at a uniform angular distance from one another in the circumferential direction.

In one development of the invention, the multi-lumen wire or an outermost one of the coaxial wires is surrounded by a shrink-on sleeve. This embodiment can advantageously be used as necessary, by virtue of the compressive effect of the shrink-on sleeve, to fix the stiffening pieces and the spacer pieces in their position in a manner secured against unintentional axial movement. This makes it possible to reliably prevent the stiffening pieces and the spacer pieces from slipping or moving away from one another axially, even if they are inserted loosely into the guide wire. The exterior of the shrink-on sleeve can moreover provide a desired surface of the guide wire, e.g. a hydrophilic or antithrombogenic surface.

In one development of the invention, the multi-lumen wire or an outermost one of the coaxial wires is provided with a hydrophilic coating or an antithrombogenic coating on the exterior. This can be advantageous for corresponding guide wire applications.

In one development of the invention, the stiffening pieces are formed from a nickel-titanium alloy or a high-grade steel material. The stiffening pieces thus formed exhibit the advantageous bending behavior known per se from these materials.

In one development of the invention, the multi-lumen wire is formed from a thermoplastic material. For this purpose, it can be produced by means of advantageously simple production methods known per se. The thermoplastic material can be e.g. a polyurethane or polyamide material.

In one development of the invention, the spacer pieces are formed from a thermoplastic material, e.g. the same material as, or a different material than, the material for the multi-lumen wire, or a thermosetting plastic material or a ceramic material. By way of example, a polytetrafluoroethylene (PTFE) material is also usable.

In one development of the invention, MR marker particles are introduced into the guide wire. The MR marker particles can be arranged e.g. in one or more of the hollow channels and/or on or in the stiffening pieces, the spacer pieces and/or the continuous tension rod and/or in the material of the multi-lumen wire and/or in a distal tip region of the guide wire adjacent to the distal end of the multi-lumen wire and/or coaxial wire construction. A continuous MR visibility of the guide wire can be realized with relatively little outlay e.g. by using the continuous tension rod and applying the MR marker particles thereon, preferably at uniform distances. If necessary, it is possible to support an X-ray visibility of the guide wire preferably in the distal end region thereof by corresponding local fitting of a spring element that is X-ray visible or of some other element that is X-ray visible.

In one development of the invention, at least one of the stiffening pieces is rounded and/or reduced in its bending stiffness at one or both ends, the reduction being achieved e.g. by corresponding tapering or heat treatment. In corresponding guide wire applications, both measures each by themselves and in combination can prevent damage to the guide wire as a result of sharp-edged end faces of the stiffening piece in a region of the guide wire that is curved during use. The end-side reduction of the bending stiffness of the stiffening piece can moreover as necessary facilitate bending of the guide wire.

Advantageous embodiments of the invention are illustrated in the drawings. The latter and further embodiments of the invention are described in greater detail below.

### BRIEF DESCRIPTION OF THE DRAWINGS

FIG. 1 shows a perspective view of a part of interest here of a guide wire having a multi-lumen wire construction comprising a central hollow channel and six eccentric hollow channels, all with a filling composed of alternating stiffening pieces and spacer pieces.

FIG. 2 shows a perspective illustration of the guide wire from FIG. 1 with transparent rendering of the multi-lumen wire to afford better visibility of the stiffening pieces and spacer pieces.

FIG. 3 shows a developed view of the hollow channels of the multi-lumen wire construction from FIG. 1, said hollow channels being filled with the stiffening pieces and spacer pieces.

FIG. 4 shows a cross-sectional view along a line IV-IV from FIG. 1.

FIG. 5 shows the cross-sectional view from FIG. 4 for a variant with a central, continuous tension rod.

FIG. 6 shows the developed view from FIG. 3 for the variant from FIG. 5.

FIG. 7 shows the cross-sectional view from FIG. 4 for a variant with four eccentric hollow channels.

FIG. 8 shows the cross-sectional view from FIG. 4 for a variant with 3 eccentric hollow channels.

FIG. 9 shows the cross-sectional view from FIG. 4 for a variant with two eccentric hollow channels.

FIG. 10 shows the cross-sectional view from FIG. 4 for a variant with a central hollow channel with a continuous tension rod and four eccentric hollow channels of larger diameter.

FIG. 11 shows the cross-sectional view from FIG. 4 for a variant with a central hollow channel with a continuous tension rod and eight eccentric hollow channels of smaller diameter.

FIG. 12 shows the cross-sectional view from FIG. 4 for a variant with a central hollow channel with a continuous tension rod and twelve eccentric hollow channels of smaller diameter.

FIG. 13 shows the cross-sectional view from FIG. 4 for a variant with a central hollow channel with a continuous tension rod and eighteen eccentric hollow channels arranged on two radii.

FIG. 14 shows a longitudinal sectional view of a part of interest here of a guide wire having a coaxial wire construction.

FIG. 15 shows a cross-sectional view along a line XV-XV from FIG. 14.

FIG. 16 shows a longitudinal sectional view of a proximal end section of a guide wire having a multi-lumen wire construction and a proximal terminating dome composed of an adhesive material.

FIG. 17 shows the longitudinal sectional view from FIG. 16 for a variant with a proximal terminating dome composed of fused multi-lumen wire material.

FIG. 18 shows a longitudinal sectional view of a distal end section of a guide wire having a multi-lumen wire construction with a conically tapered distal multi-lumen wire end.

FIG. 19 shows the longitudinal sectional view from FIG. 18 for a variant with stiffening pieces ending distally in a stepped manner.

FIG. 20 shows a side view of a stiffening piece having hemispherically rounded ends that is usable in the guide wire according to the invention.

FIG. 21 shows a side view of a stiffening piece having conically tapered and rounded ends that is usable in the guide wire according to the invention.

FIG. 22 shows a side view of a stiffening piece having conically tapered ends equipped with end balls that is usable in the guide wire according to the invention.

FIG. 23 shows a side view of a stiffening piece having conically tapered ends equipped with end paddles that is usable in the guide wire according to the invention.

### DETAILED DESCRIPTION OF THE DRAWINGS

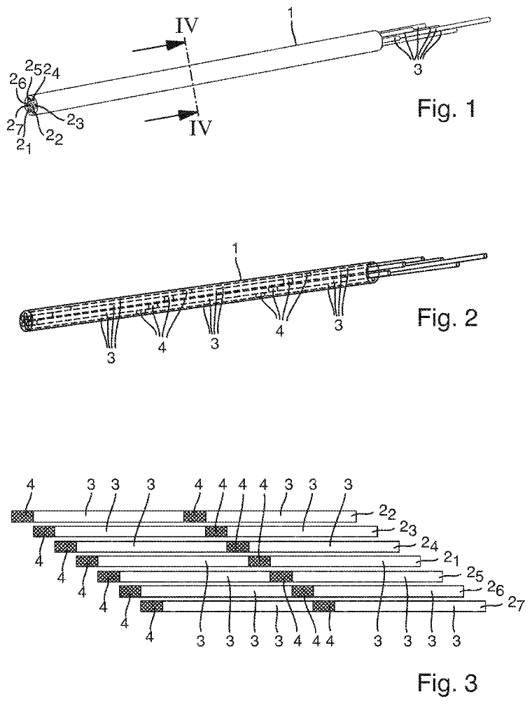

The guide wire illustrated in a representative section of interest here in FIGS. 1 to 4 is of a multi-lumen wire construction comprising a multi-lumen wire 1 composed of an electrically nonconductive plastic material. The multi-lumen wire 1 extends continuously from a proximal end region to a distal end region of the guide wire, said multi-lumen wire being shown here only with a representative partial section of its length. It has at least two separate, axially extending hollow channels 2₁, 2₂, . . . , 2ₙ, where n is an arbitrary natural number greater than one.

In the exemplary embodiment shown in FIGS. 1 to 4, the multi-lumen wire 1 includes seven hollow channels 2₁ to 2₇, specifically a central hollow channel 2₁ and six eccentric hollow channels 2₂ to 2₇, which are preferably arranged around the central hollow channel 2₁ on a common radius, alternatively in some other regular or irregular configuration. In the example shown, all the hollow channels 2₁ to 2₇ extend axially rectilinearly in the main body material of the multi-lumen wire 1 composed of plastic; in alternative embodiments, one or some of the hollow channels or all of the hollow channels extend in a helically coiled fashion. The multi-lumen wire 1 composed of the plastic material, which preferably has a relatively low bending stiffness, can be produced by any of the production methods known per se for this purpose, e.g. by means of an extrusion method.

An axially alternating sequence of rod-shaped, elastic stiffening pieces 3 and spacer pieces 4 is arranged at least in one of the hollow channels 2₁ to 2₇ of the multi-lumen wire 1. The stiffening pieces 3 consist of an electrically conductive, nonmagnetic material having a higher bending stiffness than the plastic material of the multi-lumen wire. The spacer pieces 4 consist of an electrically nonconductive material. Preferably, the stiffening pieces 3 and the spacer pieces 4 lie in the hollow channels 2₁ to 2₇ without a relatively high degree of radial play; i.e. in embodiments in this regard the external diameter of the stiffening pieces 3 and of the spacer pieces 4 is approximately equal to the internal diameter of the hollow channels 2₁ to 2₇.

Overall, therefore, the result of this multi-lumen wire construction is that the guide wire is not electrically conductive continuously from its proximal end region to its distal end region, rather only the stiffening pieces 3 form electrically conductive regions of the guide wire, which are electrically insulated from one another by the spacer pieces 4. FIGS. 1 to 4 illustrate the guide wire with a partial length comprising in each case two stiffening pieces 3 and two spacer pieces 4 in each hollow channel 2₁ to 2₇. Over its remaining length (not shown) from the proximal to the distal end region, the guide wire has a construction which corresponds to a periodic continuation of the partial length shown. In each case only the individual stiffening pieces 3 are electrically conductive, said stiffening pieces being electrically insulated from one another. Each stiffening piece 3 can consist e.g. of a solid rod or solid tube material, alternatively of a hollow rod or hollow tube material. Likewise, the spacer pieces 4 can consist e.g. of a solid material or alternatively a hollow material.

In corresponding embodiments of the guide wire, each stiffening piece 3 has a greater length than each spacer piece 4. In alternative embodiments, at least one spacer piece 4 is longer than at least one stiffening piece 3. In corresponding embodiments, the stiffening pieces 3 have an identical length among one another; in alternative embodiments, at least two stiffening pieces 3 have different lengths. Likewise, in corresponding embodiments, the spacer pieces 4 have an identical length among one another; in alternative embodiments, at least two spacer pieces 4 have different lengths. The abovementioned dimensioning specifications for the lengths of the stiffening pieces and of the spacer pieces disregard possible segments at the distal and proximal ends of the multi-lumen wire construction, which segments can arise if the stiffening pieces and spacer pieces are arranged offset in different hollow channels and at the distal and proximal ends the alternating sequence of stiffening pieces and spacer pieces is intended to terminate at the same axial height in all the hollow channels.

In advantageous embodiments, the length of each stiffening piece 3 is in the range of between 1 cm and 15 cm or more specifically between 5 cm and 10 cm. In corresponding embodiments, the length of each spacer piece 4 is in the range of between 1 mm and 5 cm or more specifically between 1 mm and 1 cm. In corresponding embodiments, the length of the respective stiffening piece 3 is greater than the length of the respective spacer piece 4 by at least a factor of 5 or more specifically by at least a factor of 10.

In advantageous embodiments, the stiffening pieces 3 and the spacer pieces 4 are arranged loosely successively in the respective hollow channel 2₁ to 2₇, i.e. are inserted loosely therein. In many applications, the stiffening pieces 3 and the spacer pieces 4 do not need to be fixed to one another and to the main body material of the multi-lumen wire 1 at the relevant hollow channel walls by connecting means, such as adhesive-bonding or welding connections. This saves associated production outlay. It suffices, if necessary, to close the hollow channels 2₁ to 2₇ at their distal end and their proximal end, such that the stiffening pieces 3 and the spacer pieces 4 cannot escape there. In order to produce the guide wire, the multi-lumen wire 1 can be prefabricated as a corresponding main body composed of plastic material, and the separately prefabricated stiffening pieces 3 and spacer pieces 4 can then be inserted alternately into the respective hollow channel 2₁ to 2₇. In an alternative embodiment, a spacer piece 4 is in each case fixed to a stiffening piece 3, e.g. by a welding or by adhesive bonding of an adhesive spot composed of an electrically insulating adhesive material, said adhesive spot functioning as a spacer piece 4 in this case, to an end face of the rod-shaped stiffening piece 3. These combination pieces each composed of a stiffening piece 3 and a spacer piece 4 can be prefabricated and then inserted into the hollow channels 2₁ to 2₇. In one variant, a respective spacer piece 4 can be fixed to both end faces of a stiffening piece 3, and these combination pieces can then be inserted alternately with stiffening pieces 3 without a premounted spacer piece 4 into the hollow channels 2₁ to 2₇.

In corresponding realizations, the stiffening pieces 3 are formed from an NiTi alloy, such as e.g. nitinol, or a high-grade steel material. The spacer pieces 4 can be formed e.g. from a thermoplastic material or a thermosetting plastic material or a ceramic material, specifically e.g. from PTFE. The stiffening pieces 3 generally serve primarily for providing a desired elastic bending stiffness or bending ability of the guide wire. In these cases, the multi-lumen wire 1 has a significantly lower bending stiffness than the stiffening pieces 3, and the spacer pieces 4 are likewise flexurally more pliant than the stiffening pieces 3 and/or do not significantly contribute to the bending stiffness of the guide wire owing to a significantly shorter length. The multi-lumen wire 1 is preferably formed from a thermoplastic material. It is optionally provided with a hydrophilic coating or an antithrombogenic coating on the exterior.

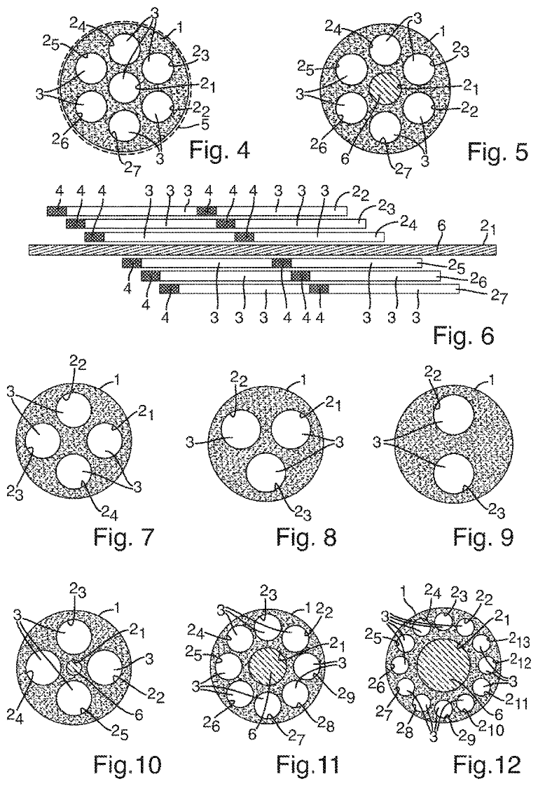

Optionally, the multi-lumen wire 1 is surrounded by a shrink-on sleeve 5, indicated in a dashed manner in FIG. 4. The shrink-on sleeve 5 can be used as necessary to compress the main body material of the multi-lumen wire 1 radially inward. This can support as necessary the axial fixing of the stiffening pieces 3 and the spacer pieces 4 in the hollow channels 2₁ to 2₇.

In corresponding embodiments, MR marker particles are introduced into the guide wire, and more specifically into the multi-lumen wire construction. Said particles can be situated e.g. in one or more of the hollow channels 2₁ to 2₇ and/or on or in the stiffening pieces 3 and/or on or in the spacer pieces 4 and/or in the material of the multi-lumen wire 1.

In advantageous embodiments of the wire of the invention, as in the exemplary embodiment in FIGS. 1 to 4, the alternating sequence of the stiffening pieces 3 and spacer pieces 4 is arranged in a plurality of the hollow channels 2₁ to 2₇, wherein the stiffening pieces 3 and spacer pieces 4 in a first of said hollow channels, e.g. in the hollow channel 2₁, are arranged axially offset relative to the stiffening pieces 3 and spacer pieces 4 in a second of said hollow channels, e.g. in the hollow channel 2₂, as is evident in particular from FIGS. 2 and 3. In the embodiment in FIGS. 1 to 4, the stiffening pieces 3 and spacer pieces 4 in one of the hollow channels 2₁ to 2₇ are offset by the length of the spacer piece 4 relative to the stiffening pieces 3 and spacer pieces 4 in another of the hollow channels 2₁ to 2₇, i.e. in the developed view in FIG. 3 the spacer pieces 4 succeed one another from one hollow channel 2₁ to 2₇ to a next without any gaps, with the exception of a possible gap between a last hollow channel in the developed view in FIG. 3, e.g. the hollow channel 2₇, and a first hollow channel, e.g. the hollow channel 2₂, at which the next sequence of spacer pieces 4 and stiffening pieces 3 then begins.

As is evident specifically from FIG. 3, in the exemplary embodiment in FIGS. 1 to 4, the spacer pieces 4 are arranged in a manner axially free of overlap in the guide wire, i.e. at any arbitrary point of the guide wire along the axial length thereof in the cross section of the guide wire there is at most one spacer piece 4 present. Since, in this example, all the hollow channels 2₁ to 2₇ are filled with the alternating sequence of stiffening pieces 3 and spacer pieces 4 without any gaps, this means, conversely, that at any arbitrary point along the axial extent of the guide wire in cross section there are always at least six stiffening pieces 3 present. Such a construction contributes to the guide wire having a very uniform stiffness or a very uniform bending moment along its axial extent.

In embodiments in which, as in the example in FIGS. 1 to 4, all the hollow channels 2₁ to 2₇ are occupied by loosely introduced stiffening pieces 3 and spacer pieces 4, tensile force loads are absorbed by the main body plastic material of the multi-lumen wire 1, i.e. this multi-lumen wire main body composed of plastic is designed in this case to provide the required tensile strength of the guide wire.

FIGS. 5 to 13 illustrate various embodiment variants of the guide wire from FIGS. 1 to 4, having a multi-lumen wire construction. Specifically, FIGS. 5 and 6 show an exemplary embodiment corresponding to that in FIGS. 1 to 4 with the sole modification that, instead of the sequence of alternating stiffening pieces 3 and spacer pieces 4, a continuous tension rod 6 composed of an electrically nonconductive plastic material of high strength is arranged in the central hollow channel 2₁.

The continuous tension rod 6 can absorb tensile forces, and so it is possible, if desired, to relieve the burden on the multi-lumen wire 1 with its main body material with regard to tensile force requirements. The continuous tension rod 6 is generally embodied as relatively flexurally pliant, such that it does not significantly influence the bending stiffness of the entire guide wire as defined by the stiffening pieces 3.

The continuous tension rod 6 can consist e.g. of a high-strength plastic material such as is known per se as a central core wire, for example, for use in guide wires. Optionally, MR marker particles are arranged on the continuous tension rod 6, e.g. on its surface. The MR marker particles can preferably be arranged continuously or at preferably regular distances over the entire or a predominant portion of the length of the tension rod 6.

In alternative embodiments (not shown), the continuous tension rod 6 is arranged in one of the eccentric hollow channels 2₂ to 2₇, or a plurality of such continuous tension rods 6 are arranged in a plurality of hollow channels chosen arbitrarily, wherein in the latter case the alternating sequence of stiffening pieces 3 and spacer pieces 4 is arranged only in one or more other hollow channels.

An embodiment illustrated in FIG. 7 differs from that in FIGS. 1 to 4 in that the multi-lumen wire 1 has only four eccentric hollow channels 2₁ to 2₄, which are preferably formed in the main body plastic material of the multi-lumen wire 1 at an identical angular distance of 90° around a longitudinal central axis of the guide wire. In this example shown, all four hollow channels 2₁ to 2₄ are provided with the alternating sequence of stiffening pieces 3 and spacer pieces 4. In alternative embodiments, one or more continuous tension rods 6 of the type explained above with regard to the exemplary embodiment in FIGS. 5 and 6 can instead be provided in one, two or three of the hollow channels 2₁ to 2₄.

An embodiment variant shown in FIG. 8 corresponds to that from FIG. 7 with the exception that only three eccentric hollow channels 2₁, 2₂, 2₃ are provided in the multi-lumen wire 1. Preferably, the three hollow channels 2₁, 2₂, 2₃ are arranged, as shown, at a uniform angular distance of 120° around a longitudinal central axis of the guide wire. In the embodiment shown, once again all the hollow channels 2₁, 2₂, 2₃ are filled with the alternating sequence of stiffening pieces 3 and spacer pieces 4. In alternative embodiments, the abovementioned continuous tension rod 6 is instead arranged in one or two of these three hollow channels 2₁, 2₂, 2₃.

In an embodiment variant illustrated in FIG. 9, the multi-lumen wire 1 has only two eccentric hollow channels 2₁, 2₂. In the embodiment shown, the respective alternating sequence of the stiffening pieces 3 and spacer pieces 4 is arranged in both hollow channels 2₁, 2₂. In an alternative embodiment, the continuous tension rod 6 is instead arranged in one of the hollow channels 2₁, 2₂.

In the exemplary embodiments in FIGS. 1 to 9, all the hollow channels 2₁ to 2₇ of the multi-lumen wire 1 have a circular cross section having the same diameter. In alternative embodiments, the hollow channels have a different cross-sectional shape, e.g. an oval, elliptic or polygonal cross section, and/or they have mutually different cross-sectional shapes and/or cross-sectional areas. Embodiment variants of this type are illustrated in FIGS. 10 to 12.

In the embodiment variant in FIG. 10, the multi-lumen wire 1 has a central hollow channel 2₁ having a smaller cross section and four eccentric hollow channels 2₂ to 2₅ having a mutually identical cross section or diameter that is larger than that of the first hollow channel 2₁. As in the example in FIG. 5, the continuous tension rod 6 is arranged in the central hollow channel 2₁, said tension rod having a smaller cross section in this case, while the alternating sequence of the stiffening pieces 3 and spacer pieces 4 is arranged in the eccentric hollow channels 2₂ to 2₅. In this case, the stiffening pieces 3 and the spacer pieces 4 have a larger cross section than the continuous tension rod 6.

In an embodiment variant shown in FIG. 11, the multi-lumen wire 1 has a central hollow channel 2₁ having a larger cross section and eight eccentric hollow channels 2₂ to 2₉ having a cross section that is smaller than that of the central hollow channel 2₁. Once again a continuous tension rod 6, here having a correspondingly larger cross section, is introduced into the central hollow channel 2₁, while the alternating sequence of stiffening pieces 3 and spacer pieces 4 is arranged in the eccentric hollow channels 2₂ to 2₉, which once again are arranged at a preferably equidistant angular distance around the central hollow channel 2₁.

In the embodiment variant illustrated in FIG. 12, the multi-lumen wire 1 includes a central hollow channel 2₁ having a relatively large cross section and twelve eccentric hollow channels 2₂ to 2₁₃ having a significantly smaller cross section by comparison therewith, said eccentric hollow channels once again being arranged at a preferably equidistant angular distance around the central hollow channel 2₁. Once again the continuous tension rod 6, here having a correspondingly large cross section, is arranged in the central hollow channel 2₁, and the eccentric hollow channels 2₂ to 2₁₃ are provided with the alternating sequence of stiffening pieces 3 and spacer pieces 4. In further alternative embodiments (not shown), the continuous tension rod 6 of the exemplary embodiments in accordance with FIGS. 10 to 12 is replaced by the alternating sequence of stiffening pieces 3 and spacer pieces 4, and/or one or more continuous tension rods 6 are arranged in one or more of the eccentric hollow channels, wherein the alternating sequence of the stiffening pieces 3 and spacer pieces 4 is in each case still arranged in at least one of the hollow channels.

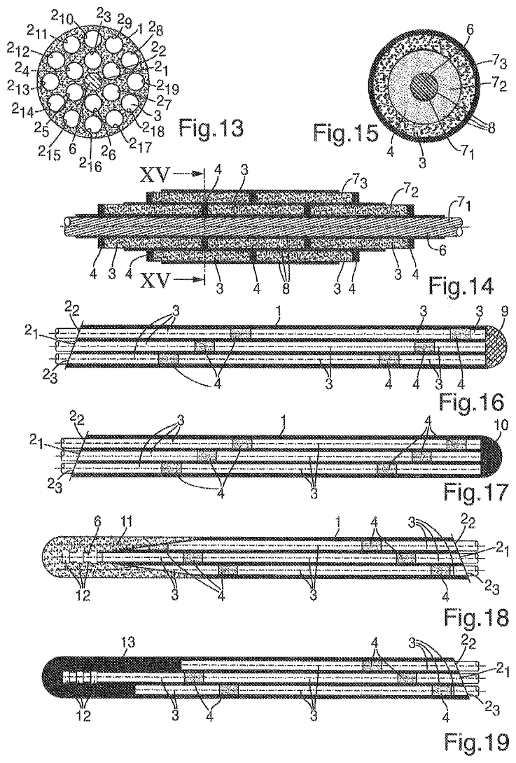

In an embodiment illustrated in FIG. 13, the multi-lumen wire 1 has a central hollow channel 2₁, a first group of eccentric hollow channels, e.g. as shown six such hollow channels 2₂ to 2₇, which are arranged on a first radius around the central hollow channel 2₁, and a second group of eccentric hollow channels, which is arranged on a second radius around the first group of hollow channels, e.g. as shown twelve such hollow channels 2₈ to 2₁₉.

In the example shown in FIG. 13, the abovementioned continuous tension rod 6 is arranged in the central hollow channel 2₁, while the remaining hollow channels 2₂ to 2₁₉ are occupied by the alternating sequence of stiffening pieces 3 and spacer pieces 4. In alternative embodiments, the continuous tension rod 6 in the central hollow channel 2₁ is replaced by the sequence of stiffening pieces 3 and spacer pieces 4 and/or in one or more of the eccentric hollow channels 2₂ to 2₁₉ the continuous tension rod 6 is arranged instead of the alternating sequence of stiffening pieces and spacer pieces 4. In the example shown, all the hollow channels 2₁ to 2₁₉ have an identical, circular cross section; in alternative embodiments, at least two of the hollow channels 2₁ to 2₁₉ differ in their cross-sectional shape and/or in their cross-sectional area. Generally, a larger number of hollow channels and thus a larger number of stiffening pieces 3 and spacer pieces 4 and/or continuous tension rods 6 accommodated therein is advantageous with regard to achieving a uniform bending behavior of the guide wire.

FIGS. 14 and 15 illustrate a guide wire having a coaxial wire construction. The latter includes at least two coaxial wires 7₁, . . . , 7ₙ, where n is an arbitrary natural number greater than one, that are arranged in a manner lying coaxially one inside another and extend continuously from a proximal end region to a distal end region of the guide wire. These are specifically three coaxial wires 7₁, 7₂, 7₃ in the example shown in FIGS. 14 and 15, and only two or more than three coaxial wires in alternative embodiments.

At least one of the coaxial wires 7₁ to 7ₙ is constructed from an axially alternating sequence of rod-shaped, elastic stiffening pieces 3 composed of an electrically conductive, nonmagnetic material and electrically nonconductive spacer pieces 4. The stiffening pieces 3 correspond in terms of material, shape and function to those of the abovementioned guide wires having a multi-lumen wire construction, and the spacer pieces 4 likewise correspond in terms of material, shape and function to those of the abovementioned guide wires having a multi-lumen wire construction.

In the exemplary embodiment in FIGS. 14 and 15, a first coaxial wire 7₁ forms a central coaxial wire functioning as a core wire. The central coaxial wire 7₁ is formed from solid material, alternatively from a hollow tube material. In the example shown, the central coaxial wire 7₁ is specifically formed by a tension rod 6 of the type explained above with regard to the guide wires having a multi-lumen wire construction, said tension rod extending continuously from the proximal end region to the distal end region of the guide wire, which encompasses an embodiment of the tension rod 6 composed of solid or hollow rod material.

In the exemplary embodiment shown in FIGS. 14 and 15, the central coaxial wire 7₁ is surrounded coaxially by a second coaxial wire 7₂, which is in turn surrounded coaxially by a third coaxial wire 7₃. Overall said coaxial wire construction results as a tube-in-tube construction, wherein the second and third coaxial wires 7₂, 7₃ each have a hollow tube shape so as to accommodate the radially inwardly adjacent coaxial wire in the interior. An electrically insulating coating or a shrink-on sleeve material 8 is preferably provided between the mutually adjoining coaxial wires 7₁, 7₂, 7₃. Such a coating or such a shrink-on sleeve material 8 is optionally additionally provided on the exterior of the outermost coaxial wire 7₃, wherein a hydrophilic or antithrombogenic coating can preferably also be involved in this last-mentioned case.

In the example shown in FIGS. 14 and 15, the central, second coaxial wire 7₂ and the outer, third coaxial wire 7₃ are each formed from the axially alternating sequence of stiffening pieces 3 and spacer pieces 4. For this purpose, the stiffening pieces 3 and the spacer pieces 4 each have a corresponding hollow tube shape or ring shape. In order to produce the guide wire, e.g. individual hollow-tube-shaped or ring-shaped stiffening pieces 3 and spacer pieces 4 can be prefabricated and threaded onto the central coaxial wire 7₁ or the already formed, radially inner coaxial wire construction comprising the central, inner coaxial wire 7₁ and the second, central coaxial wire 7₂.

For the dimensioning and the arrangement of the stiffening pieces 3 and the spacer pieces 4, in the case of the guide wire having the coaxial wire construction, the embodiment variants explained above with regard to the guide wires having a multi-lumen wire construction can be realized in a corresponding manner, and so in respect thereof reference can be made to the explanations above. This applies in particular with regard to material selection and length extent for the stiffening pieces 3, on the one hand, and the spacer pieces 4, on the other hand. The coaxial wire construction makes it possible to achieve a bending behavior that is very uniform in a direction-dependent manner for the guide wire. In the case of the coaxial wire construction, too, in corresponding embodiments, the stiffening pieces 3 and the spacer pieces 4, if they are provided for a plurality of coaxial wires, in the case of at least one of said coaxial wires are preferably arranged axially offset relative to those in the case of at least one of the other coaxial wires. What is realized in the example shown in FIGS. 14 and 15 is a central offset of the stiffening pieces 3 and spacer pieces 4 in their arrangements for the second and third coaxial wires 7₂, 7₃. Furthermore, in corresponding embodiments, the spacer pieces 4 are arranged in a manner axially free of overlap in the guide wire, as is also the case in the example in FIGS. 14 and 15.

In embodiment variants (not shown) of the coaxial wire construction in FIGS. 14 and 15, the guide wire consists only of two coaxial wires lying one inside the other or of more than three coaxial wires lying one inside another. In alternative embodiments to the exemplary embodiment in FIGS. 14 and 15, the alternating sequence of the stiffening pieces 3 and the spacer pieces 4 is replaced by the continuous tension rod 6 in one of the two relevant coaxial wires 7₂, 7₃, said tension rod being formed with a suitable hollow rod shape in this case. In a further alternative embodiment, the central coaxial wire 7₁ is formed by the alternating sequence of stiffening pieces 3 and spacer pieces 4 instead of by the tension rod 6. In this case, the stiffening pieces 3 and spacer pieces 4 are preferably formed from solid material.

In further embodiments, the multi-lumen wire construction and the coaxial wire construction are combined with one another. In one realization in this regard, the guide wire has the coaxial wire construction of the type in FIGS. 14 and 15 comprising two or an arbitrary larger number of coaxial wires 7₁ to 7ₙ, wherein the central coaxial wire 7₁ is formed by the multi-lumen wire 1 composed of electrically nonconductive plastic material with the at least two axial hollow channels 2₁ to 2ₙ, wherein the alternating sequence of stiffening pieces 3 and spacer pieces 4 is situated in at least one of said hollow channels 2₁ to 2ₙ of the multi-lumen wire 1. In alternative realizations, one or more of the surrounding coaxial wires 7₂ to 7ₙ are formed by the multi-lumen wire 1, which in this case is provided in a corresponding hollow tube embodiment and has the axially extending hollow channels 2₁ to 2ₙ in its hollow tube material, wherein once again at least one of said hollow channels 2₁ to 2ₙ contains the alternating sequence of stiffening pieces 3 and spacer pieces 4.

Generally, in these combination embodiments, the guide wire can consist of a plurality of coaxial wires, of which at least one is formed by the multi-lumen wire 1 with the alternating sequence of stiffening pieces 3 and spacer pieces 4 in at least one of its hollow channels 2₁ to 2ₙ and optionally with the continuous tension rod 6 in any remaining hollow channels and the remaining coaxial wires are formed by the alternating sequence of stiffening pieces 3 and spacer pieces 4 or by the continuous tension rod 6.

A guide wire illustrated in FIG. 16 has a multi-lumen wire construction of the type explained above, wherein the multi-lumen wire 1 is terminated at its proximal end by a hemispherical dome 9 composed of an adhesive material. The terminating dome 9 proximally closes the hollow channels 2₁ to 2ₙ of the multi-lumen wire 1 and thereby also prevents possible proximal escape of the stiffening pieces 3 and spacer pieces 4 accommodated in the hollow channels 2₁ to 2ₙ.

FIG. 17 illustrates an embodiment variant of the guide wire from FIG. 16. In this variant, a hemispherical, proximal terminating dome 10 is likewise provided, but in this case it consists of fused plastic material of the multi-lumen wire 1. Therefore, no additional adhesive material need be applied for this realization. Otherwise, the terminating dome 10 in FIG. 17 corresponds to the proximal terminating dome 9 in FIG. 16 in terms of shape and function.

FIG. 18 illustrates, in a distal end section of interest here, a guide wire having a multi-lumen wire construction of the abovementioned type, in which the multi-lumen wire 1 together with the stiffening pieces 3 and spacer pieces 4 introduced in the hollow channels 2₁ to 2ₙ is embodied as conically tapered at its distal end, e.g. by means of a corresponding grinding process. This results in a desired lower bending stiffness of the distal end section for this guide wire.

In a manner known per se, a distal end cap 11 composed of a suitable, flexurally pliant filling material is joined to the conically tapered end part of the multi-lumen wire 1. A constant diameter of the guide wire as far as the distal termination thereof is thereby maintained. Optionally, one or more MR markers 12 are arranged at the distal tip end of the multi-lumen wire 1, as shown, or alternatively on or in the material of the distal end cap 11. In advantageous embodiments of this type, the multi-lumen wire 1 has a central hollow channel with a continuous tension rod 6 introduced therein. Preferably, in these cases the tension rod 6 projects distally beyond the conical part of the distal tip end of the multi-lumen wire 1 and carries the MR markers 12 and/or contributes to a secure support of the end cap 11 and/or to ensuring a sufficient tensile strength of the guide wire including in this distal terminating region. In optional embodiments (not shown), in the distal end section of the guide wire it is possible to provide an element that improves X-ray visibility, e.g. a helical spring pushed onto the distal end of the multi-lumen wire 1 or of the central tension rod or embedded into the distal end cap filling material.

FIG. 19 shows a variant of the guide wire from FIG. 18, in which the low bending stiffness of the distal guide wire end section compared with the proximally adjacent guide wire region is realized by stepped introduction of the alternating sequence of stiffening pieces 3 and spacer pieces 4 into the different hollow channels 2₁ to 2ₙ instead of by conical tapering of the multi-lumen wire 1. This means that the last stiffening pieces 3 distally in each case in the affected hollow channels 2₁ to 2ₙ end distally at different axial heights.

Once again a distal end cap 13 composed of a suitable filling material functions as a distal termination of the guide wire, wherein a distal residual length in the affected hollow channels that remains as a result of the stepped arrangement of the last stiffening pieces 3 distally can also be closed by said filling material. The filling material for the distal terminating cap 13 can correspond to, or be different than, that for the distal terminating cap 11 in the variant from FIG. 18. In a corresponding realization, the filling material for the distal terminating cap 13 is formed from the plastic material of the multi-lumen wire 1. Optionally, the multi-lumen wire 1 can once again have a central hollow channel with the tension wire 6 introduced therein, which then, analogously to the exemplary embodiment from FIG. 18, preferably forms the wire element extending furthest toward the front distally and provides for the required tensile strength of the distal terminating region and, if necessary, can function as a carrier of the MR markers 12.

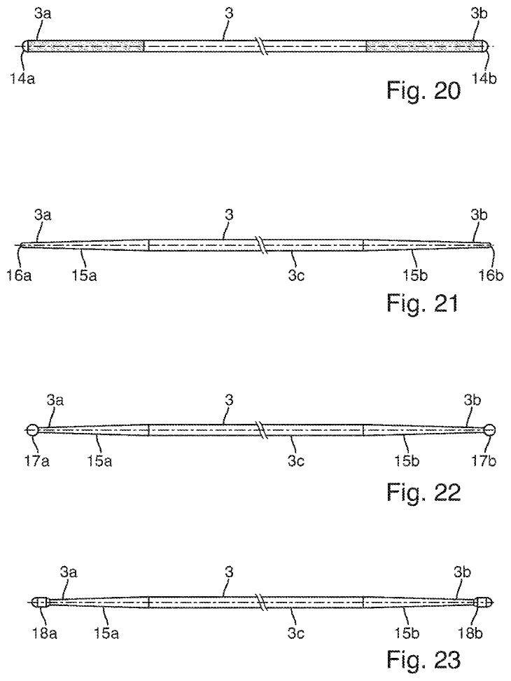

As already mentioned, the stiffening pieces 3 consist of a corresponding solid rod or solid tube material or hollow rod or hollow tube material. For this purpose, they can be produced e.g. by cutting to length a corresponding solid rod or solid tube or hollow rod or hollow tube. In corresponding embodiments, the respective stiffening piece 3 can be used in an end-treated realization, according to which it is rounded at one of its two ends or at both ends and/or is reduced in terms of its bending stiffness. As a result, if necessary, it is possible to influence the bending behavior of the guide wire in a desired manner and/or to prevent damage to the guide wire particularly in the case of relatively high curvature loads of the guide wire. FIGS. 20 to 23 illustrate four exemplary examples in this respect, in which the stiffening piece 3 is end-treated in each case on both sides. In alternative embodiments, the stiffening piece 3 is end-treated in this way only at one of its two ends. In corresponding embodiments of the guide wire, at least one of the stiffening pieces 3 used in it is end-treated, preferably a plurality of its stiffening pieces 3 including the case where all of its stiffening pieces 3 are end-treated.

In the exemplary embodiment in FIG. 20, the stiffening piece 3 is rounded at both of its ends 3a, 3b in each case by virtue of the fact that it terminates there with a hemispherical terminating dome 14a, 14b at the end side. The terminating dome 14a, 14b can be formed in a simple manner in terms of production engineering e.g. by fusing of the stiffening piece material at the end side of the respective end 3a, 3b of the stiffening piece 3. By virtue of the terminating domes 14a, 14b, the stiffening piece 3 does not end axially with an abrupt end marginal edge, as a result of which it is possible to prevent such an end marginal edge from damaging or piercing a surrounding material, such as a surrounding hollow channel material or an outer skin of the guide wire, if the stiffening piece 3 is situated with its relevant end 3a, 3b in a region in which the guide wire is curved to a relatively great degree.

FIG. 21 illustrates an exemplary embodiment in which the stiffening piece 3 is conically tapered at both of its ends 3a, 3b; i.e. in each case a conically tapered end region 15a, 15b is formed, the diameter of which decreases from a constant diameter of the stiffening piece 3 in a central region 3c toward the associated end face, preferably in a continuously variable manner, alternatively in one or more steps. Optionally, as in the example shown, the conically tapered end region 15a, 15b is rounded at its end face termination, e.g. once again with a hemispherical terminating dome 16a, 16b. As a result of this progressive end-side tapering, the bending stiffness of the stiffening piece 3 is reduced at the corresponding end 3a, 3b. As a result, the stiffening piece 3 in this region can more easily yield to or follow a bend or curvature of the guide wire, as a result of which the stiffening piece 3 presses to a lesser extent counter to the direction of curvature radially outwardly against adjoining material, such as a multi-lumen wire material of the guide wire or an outer skin of the guide wire, which prevents possible harm or damage to said material caused by the stiffening piece 3 particularly in curved sections of the guide wire. A contribution is made to the latter, moreover, by the end-side rounding of the conically tapering regions 15a, 15b by the terminating domes 16a, 16b.

FIG. 22 illustrates an exemplary embodiment which corresponds to that from FIG. 21 in the formation of the conically tapering regions 15a, 15b and differs therefrom in that the end face termination of the conically tapering regions 15a, 15b is formed respectively by a rounding terminating ball 17a, 17b, the diameter of which is greater than the adjoining minimum diameter of the conically tapering region 15a, 15b. This end-side thickening can prevent possible damage to the guide wire in the event of relatively high curvature thereof, in particular prevent the tapered end of the conically tapering region 15a, 15b from damaging or piercing a surrounding material, such as a sheathing or outer skin of the guide wire, if the guide wire is curved in this region.

The exemplary embodiment shown in FIG. 23 with the conically tapering regions 15a, 15b of the stiffening piece 3 corresponds to the examples in FIGS. 21 and 22, wherein in this case the respective conically tapering region 15a, 15b is not embodied as far as the end face of the stiffening piece 3, but rather ends somewhat at a distance therefrom and transitions via an axially short, conically widening region into the original diameter of the rod blank used for producing the stiffening piece 3, in order then to terminate again with a hemispherical terminating dome analogously to the exemplary embodiment from FIG. 20, thus resulting overall in a respective paddle-shaped end termination 18a, 18b for the stiffening piece 3. In this case, too, a reduced bending stiffness of the stiffening piece 3 at the end side is advantageously combined with end rounding that affords protection against damage.

Optionally, in each of the embodiments illustrated in FIGS. 20 to 23, a heat treatment of one or both ends 3a, 3b of the stiffening piece 3 can additionally be provided, in particular by soft annealing or an increase of the so-called AF temperature of superelastic alloys such as NiTi alloys. Said heat treatment likewise brings about a reduction of the bending stiffness of the stiffening piece 3 at its relevant end 3a, 3b or end region.

As made clear by the exemplary embodiments shown and explained above, the invention provides, in a very advantageous way, a guide wire for medical MR applications which is optimized in terms of its behavior under MR conditions and, in particular, avoids undesired heating effects under MR conditions. The guide wire can be produced with comparatively low production outlay and affords a high functional reliability. By means of suitable system design, the guide wire can as necessary be optimally coordinated with the respective application in terms of its bending behavior or in terms of its flexibility.

1.-14. (canceled)

15. A guide wire configured for use in medical magnetic resonance applications, comprising:
a multi-lumen wire composed of an electrically nonconductive plastic material, said multi-lumen wire extending continuously from a proximal end region to a distal end region of the guide wire and having at least two separate, axially extending hollow channels, wherein
an axially alternating sequence of rod-shaped, elastic stiffening pieces composed of an electrically conductive, nonmagnetic material having higher bending stiffness than the plastic material of the multi-lumen wire, and electrically nonconductive spacer pieces, is arranged at least in one of the hollow channels of the multi-lumen wire.

16. A guide wire configured for use in medical magnetic resonance applications, comprising:
coaxial wires arranged coaxially one inside another and extending continuously from a proximal end region to a distal end region of the guide wire, wherein
at least one of the coaxial wires is constructed from an axially alternating sequence of rod-shaped, elastic stiffening pieces composed of an electrically conductive, nonmagnetic material and electrically nonconductive spacer pieces.

17. The guide wire as claimed in claim 15, further comprising:
coaxial wires arranged coaxially one inside another and extending continuously from a proximal end region to a distal end region of the guide wire, wherein
at least one of the coaxial wires is constructed from an axially alternating sequence of rod-shaped, elastic stiffening pieces composed of an electrically conductive, nonmagnetic material and electrically nonconductive spacer pieces.

18. The guide wire as claimed in claim 15, further comprising at least one of the following dimensional features:
each stiffening piece has a greater length than each spacer piece,
the length of each stiffening piece is in the range of between 1 cm and 15 cm, and
the length of each spacer piece is in the range of between 0.1 mm and 5 cm.

19. The guide wire as claimed in claim 15, wherein
the stiffening pieces and the spacer pieces are arranged loosely successively.

20. The guide wire as claimed in claim 15, wherein
a continuous tension rod composed of an electrically nonconductive plastic material is arranged at least in one of the hollow channels.

21. The guide wire as claimed in claim 20, wherein
the hollow channels include a central hollow channel, in which the continuous tension rod composed of electrically nonconductive plastic material is arranged.

22. The guide wire as claimed in claim 16, wherein
at least one of the coaxial wires is formed by a continuous tension rod composed of an electrically nonconductive plastic material.

23. The guide wire as claimed in claim 22, wherein
the coaxial wires include a central coaxial wire, which is formed by the continuous tension rod composed of an electrically nonconductive plastic material.

24. The guide wire as claimed in claim 15, wherein
the hollow channels include a central hollow channel, in which the alternating sequence of the stiffening pieces and spacer pieces is arranged.

25. The guide wire as claimed in claim 15, wherein
for a plurality of the hollow channels, the alternating sequence of the stiffening pieces and spacer pieces is arranged, and
the stiffening pieces and spacer pieces in a first of said hollow channels are arranged axially offset relative to the stiffening pieces and spacer pieces in a second of said hollow channels.

26. The guide wire as claimed in claim 25, wherein
the spacer pieces are arranged in a manner axially free of overlap in the guide wire.

27. The guide wire as claimed in claim 15, wherein
the hollow channels include a plurality of eccentric hollow channels, in which the alternating sequence of the stiffening pieces and spacer pieces is arranged.

28. The guide wire as claimed in claim 15, wherein
the multi-lumen wire is surrounded by a shrink-on sleeve or is provided with a hydrophilic coating or an antithrombogenic coating on the exterior.

29. The guide wire as claimed in claim 15, further comprising at least one of the following features:
the stiffening pieces are formed from a nickel-titanium alloy or a high-grade steel material,
the multi-lumen wire is formed from a thermoplastic material,
the spacer pieces are formed from a thermoplastic material or a thermosetting plastic material or a ceramic material, and
magnetic resonance marker particles are introduced into the guide wire.

30. The guide wire as claimed in claim 15, wherein
at least one of the stiffening pieces is rounded or reduced in its bending stiffness at one or both ends.

31. The guide wire as claimed in claim 16, further comprising at least one of the following dimensional features:
each stiffening piece has a greater length than each spacer piece,
the length of each stiffening piece is in the range of between 1 cm and 15 cm, and
the length of each spacer piece is in the range of between 0.1 mm and 5 cm.

32. The guide wire as claimed in claim 16, wherein
the stiffening pieces and the spacer pieces are arranged loosely successively.

33. The guide wire as claimed in claim 16, wherein
the coaxial wires include a central coaxial wire, which is formed by the alternating sequence of the stiffening pieces and spacer pieces.

34. The guide wire as claimed in claim 16, wherein
for a plurality of the coaxial wires, the alternating sequence of the stiffening pieces and spacer pieces is arranged, and
the stiffening pieces and spacer pieces in a first of said coaxial wires are arranged axially offset relative to the stiffening pieces and spacer pieces in a second of said coaxial wires.
